(19) United States
(12) Patent Application Publication
(10) Pub. No.: US 2019/0298477 A1
(43) Pub. Date: Oct. 3, 2019

(54) Tissue Handling Skills Trainer

(71) Applicant: Virginia Commonwealth University, Richmond, VA (US)

(72) Inventors: Sindora Baddam, Richmond, VA (US); Millie Shah, Burtonsville, MD (US); Albara Elshaer, Richmond, VA (US); Jennifer Wayne, Richmond, VA (US); Jennifer Rhodes, Midlothian, VA (US); Veronica Peterkin, Richmond, VA (US)

(21) Appl. No.: 16/365,866

(22) Filed: Mar. 27, 2019

Related U.S. Application Data
(60) Provisional application No. 62/650,616, filed on Mar. 30, 2018.

(51) Int. Cl. A61B 90/00; G09B 23/30; A61B 17/28; G09B 23/28

(52) U.S. Cl. CPC A61B 90/06; G09B 23/30; A61B 34/76; G09B 23/285; A61B 17/28

(57) ABSTRACT

A tissue handling skills trainer is provided to teach safe biological tissue manipulation using objective pressure-sensing feedback. The trainer may include a forceps body having two elongated members hinged at one end, at least one pressure sensor arranged on at least one of the elongated members, a microcontroller electronically connected to the at least one pressure sensor, and an audio and/or visual indicator electronically connected to the microcontroller, wherein the audio and/or visual indicator is triggered when the pressure sensor detects a pressure that exceeds a predetermined threshold. Methods of using the trainer are also provided.

### FIELD OF THE INVENTION

Embodiments of the invention provide a training device that improves a user's tactile and proprioception skills for the development of gentle tissue handling skills.

### BACKGROUND OF THE INVENTION

Tissue handling is fundamental to performing mechanical tasks of suturing and surgery on live humans or animals. All surgical procedures require proper tissue handling technique. Perhaps the most critical element is to handle the tissue with an appropriate touch, that is applying the least amount of force necessary to complete the task via the forceps. Whether the procedure involves skin, muscle, blood vessels or hollow organs, excessive force can crush or destroy the tissue. Even when the macrostructure of the tissue is not overwhelmed, blood flow may be jeopardized resulting in ischemia and poor healing. This both increases the risk of infection and breakdown of the wound or suture line. A delicate touch with tissue manipulation is the sina qua non of a good technical surgeon.

While the mechanics of suturing can be taught, there is no available simulation product designed to specifically teach tissue handling skills. Thus, learning the techniques of proper tissue handling is currently a trial and error experience, save for the post hoc observation of tissue damage. As it is the non-dominant hand which holds the forceps to grasp the tissue while the dominant hand performs the task (e.g. suturing), attention is preferentially directed towards the dominant, working hand. It generally takes several of years of experience with suturing, under supervision with directed feedback, to begin to develop a “light touch” in the non-dominant hand in particular. Learners not in surgical training programs afforded such close supervision may never garner the feedback to allow development of advance tissue handling skills.

### SUMMARY OF THE INVENTION

The tissue handling skills trainer, according to embodiments of the invention, is a device that utilizes an objective feedback mechanism to enhance a user's tissue handling skills and thus prevents unnecessary injury to the tissue.

An aspect of the disclosure provides a tissue handling skills trainer, comprising a forceps body having two elongated members hinged at one end, at least one pressure sensor arranged on at least one of the elongated members, a microcontroller electronically connected to the at least one pressure sensor, and an audio and/or visual indicator electronically connected to the microcontroller, wherein the audio and/or visual indicator is triggered when the pressure sensor detects a pressure that exceeds one or more predetermined thresholds. The thresholds may be defined by the trainer. The device may also include a digital display for the threshold selected and the time count exceeding the threshold in a given training session, and a reset button to reconfigure the settings for the next training session. The pressure sensor may be arranged on an outer and/or inner surface of at least one of the elongated members.

In some embodiments, the pressure sensor is a piezoelectric sensor. In some embodiments, the trainer includes both an audio and visual indicator. In some embodiments, the trainer further comprises a digital display operably connected to the microcontroller. In some embodiments, the trainer further comprises a battery operably connected to the microcontroller. In other embodiments, the trainer is powered via a wall outlet.

Another aspect of the invention provides a method for improving tissue handling skills of a user, comprising the step of using a tissue handling skills trainer as described herein to grasp a material. In some embodiments, the material is a biological or simulated biological tissue. In some embodiments, the tissue handling skills trainer is used while the user is performing a suturing or other surgical procedure.

Additional features and advantages of the invention will be set forth in the description below, and in part will be apparent from the description, or may be learned by practice of the invention. The advantages of the invention can be realized and attained by the exemplary structure particularly pointed out in the written description and claims hereof as well as the appended drawings.

### BRIEF DESCRIPTION OF THE DRAWINGS

FIG. 1. A forceps body having pressure sensors attached to an outer surface of an elongated member according to some embodiments of the disclosure.

FIG. 2. A forceps body having pressure sensors attached to an inner surface of an elongated member and connected to a microcontroller according to some embodiments of the disclosure.

FIG. 3. A forceps body having pressure sensors attached to an outer surface of an elongated member and connected to a microcontroller according to some embodiments of the disclosure.

FIG. 4. A flow-chart demonstrating an exemplary method according to some embodiments of the disclosure.

### DETAILED DESCRIPTION

A system as described herein focuses on developing the manual dexterity and attention skills of a user with objective feedback to improve handling of biological tissue. Users are alerted when the force they are applying to a surgical instrument is excessive and could unnecessarily damage tissue. Embodiments of the invention provide a tissue handling skills trainer comprising:

a forceps body having two elongated members hinged at one end, at least one pressure sensor arranged on at least one of the elongated members, a microcontroller electronically connected to the at least one pressure sensor, and an audio and/or visual indicator electronically connected to the microcontroller, wherein the audio and/or visual indicator is triggered when the pressure sensor detects a pressure that exceeds one of a trainer-defined predetermined thresholds.

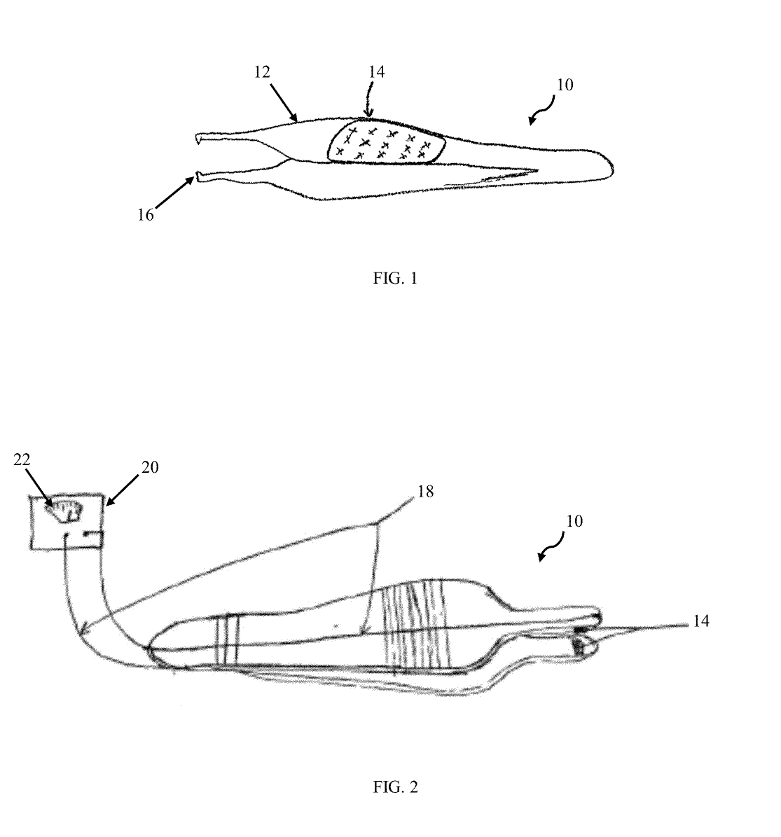

With reference to FIGS. 1 and 2, the forceps body 10 may be shaped similarly to any type of forceps used in a surgical or clinical setting, such as Adson forceps and Debakey forceps. Such forceps include two elongated members 12 that are hinged at one end forming a substantially U-shaped body. Tension at the hinged end holds the grasping ends 16 apart. The grasping ends 16 may have smooth tips, cross-hatched tips, or serrated tips.

The application of pressure to an external surface of the elongated members 12 brings the non-hinged ends of the forceps 10 together to grasp biological tissue or other materials. Such pressure is typically applied around a midpoint or more towards the grasping end of each elongated member 12 via a user's fingers. The forceps 10 may be held between the thumb and two or three fingers of one hand. The forceps 10 are typically sized to accommodate the hand of a user. In some embodiments, the forceps 10 are sized for the average adult (or average adult female or average adult male).

The forceps body 10 may be fabricated from any material suitable for use in a medical environment, such as plastic or metal materials. For example, the forceps 10 described herein may be made of high-grade carbon steel, high-quality stainless steel, chromium, and vanadium alloys to provide durability of edges and freedom from rust. Such materials are also able to withstand repeated sterilization. Lower-quality steel may also be used to produce forceps 10 as described herein as such forceps 10 may be used only for training purposes outside of a live patient situation. In some embodiments, the forceps 10 are made of plastic.

In some embodiments, one or more pressure sensors 14 may be attached to a superior surface of the forceps 10, e.g. where the index and middle fingers of a user rest and/or where the thumb of a user rests. The pressure of the fingers to the pressure sensor 14 on the forceps 10 is recorded and calibrated to match the force applied to the tissue by the forceps. Forceps 10 such as Adson forceps often have ribbed areas where the user's fingers rest. Attachment of a pressure sensor 14 to this area can be attained by any means such as using a glue that fills the entire ribbed area or tape for a more secure attachment.

In some embodiments, one or more pressure sensors 14 may be attached to an inner surface of the forceps 10. In some embodiments, one or more pressure sensors may 14 be attached to an inner surface of the grasping ends 16 of the forceps 10 so that the force on the material being grasped is directly measured.

Pressure sensors 14 (also referred to as force sensors) compatible with the device described herein are known in the art and include, but are not limited to, a piezoelectric force sensor, a piezoresistive strain gauge sensor, a capacitive sensor, an electromagnetic sensor, a strain-gauge sensor, an optical sensor, and a potentiometric sensor. Pressure sensors as defined herein may also encompass strain sensors or transducers which may be arranged anywhere along the length of the outer or inner surface of the elongated member.

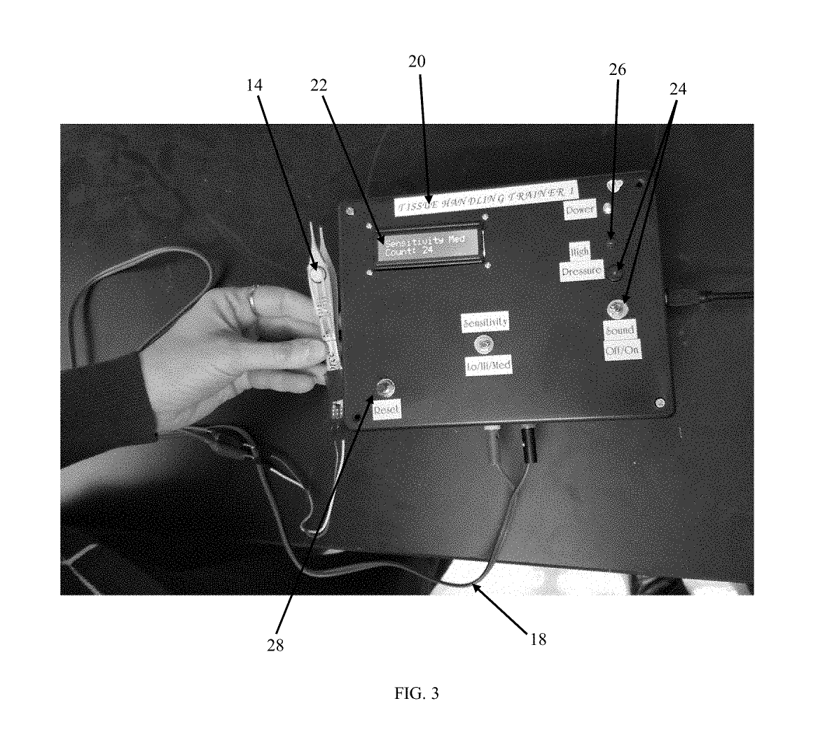

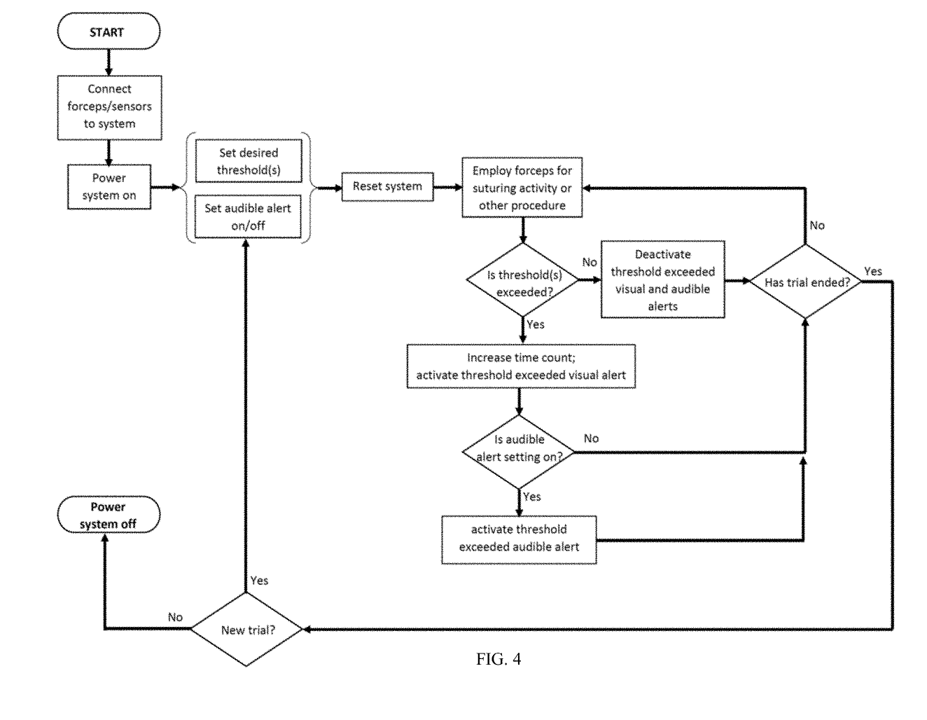

With reference to FIGS. 2 and 3, a control logic board 20, containing a microcontroller such as an Arduino microcontroller, is electronically connected, e.g. via wires 18, to the one or more pressure sensors 14. The logic board 20 is configured to receive a pressure reading from the pressure sensor(s) 14 and for carrying out the method of comparing the pressure reading to one or more threshold values and powering audio, visual, and/or tactile indicators when a threshold is exceeded. A flow-chart demonstrating an exemplary method is depicted in FIG. 4. The trainer may be battery operated. In other embodiments, the trainer is powered via a wall outlet.

The control logic board 20 may be configured to continuously measure the output from the pressure sensor(s) 14 while bearing down on the forceps, compare this force to a desired threshold, and power audio and/or visual indicators (e.g. LED) when the threshold is exceeded. The user is alerted allowing them to decrease the force, and once below the desired threshold, the alerts cease, confirming the user's action, thus constituting a closed feedback loop.

In some embodiments, there may be two stages of alerts: a safe range and a danger range. These ranges may be calibrated to the amount of pressure required to appropriately manipulate live biological tissue, e.g. human tissue. In some embodiments, the danger range may be calibrated to the amount of pressure required to appropriately manipulate live human tissue, particularly integument. In some embodiments, the device includes a third cautionary range stage that has a threshold in between that of the safe and danger ranges. The cautionary range may begin from the pressure required to manipulate the skin up until the threshold value of which risks injury to the skin. The threshold levels may be modified by adjusting the values set in the program for the microcontroller. In some embodiments, the system may provide a numeric time count that the user exceeds the threshold in a given training session. In some embodiments, the system provides a numeric count of the number of times the threshold is exceeded in a given session. In some embodiments, the system includes a digital display 22 which provides, e.g. the numeric time count, the numeric count, the sensitivity threshold, and/or any other relevant information.

In some embodiments, the device is configured to have three different skill levels to permit the trainee to continue to improve on their skills wherein each skill level has a different set of threshold values for the safe, cautionary, and danger ranges. The three different skill levels may be designated as novice, intermediate, and advanced levels, with decreasing thresholds to further refine skills of more advanced users. It is contemplated that more or less than three thresholds or skill levels may be used.

A trainer as disclosed herein may also include a reset button 28 to reconfigure the settings for the next training session.

The audible signal 24 may be a chirp, beep, siren, buzzer, or other sound signal. The audible signal may be emitted, e.g. through a speaker located on the forceps or wired to it or wirelessly connected. In some embodiments, the audible signal 24 is triggered when the user exceeds a predetermined pressure threshold and ceases when the pressure decreases below the threshold. In some embodiments, different audible signals (e.g. different sound types or different intensities) are emitted at different threshold levels as described herein.

The visual signal or indicator 26 may be, e.g. a strobe, flash, LED light or other light emitter. The visual signal may be emitted, e.g. through a visual indicator located on the forceps or wired to it or wirelessly connected. In some embodiments, the visual signal 26 is triggered when the user exceeds a predetermined pressure threshold and ceases when the pressure decreases below the threshold. In some embodiments, the different visual signals (e.g. different color lights or different flashing rates) are emitted at different threshold levels as described herein.

In some embodiments, the system emits both an audible signal and a visual signal. In some embodiments, the system emits a tactile signal, such as a vibration via a shaker attached to the forceps.

The system may alternatively, or in conjunction with generating one or both of audible and visual signals, generate a communication signal in response to the pressure applied to the forceps exceeding a predetermined threshold. The communication signal may be via a wireless signal, such as Bluetooth, RF, infrared or other wireless communication for triggering an alarm on a nearby or remote receiver, such as a computer, tablet, or mobile phone.

A system as described herein may be used to teach tissue handling skills to a variety of individuals including medical students, other medical professionals (physician assistants, nurse practitioners, surgical technologists, residents, etc.), as well as veterinary care providers, on a variety of simulated tissues including skin, hollow organ and blood or lymphatic vessels as well as on biological materials. The system could also be useful for the practicing surgeon to relearn, refine or improve gentle tissue handling skills.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A tissue handling skills trainer, comprising:
a forceps body having two elongated members hinged at one end,
at least one pressure sensor arranged on at least one of the elongated members,
a microcontroller electronically connected to the at least one pressure sensor, and
an audio and/or visual indicator electronically connected to the microcontroller, wherein the audio and/or visual indicator is triggered when the pressure sensor detects a pressure that exceeds a predetermined threshold.

2. The tissue handling skills trainer of claim 1, wherein the pressure sensor is arranged on an outer surface of at least one of the elongated members.

3. The tissue handling skills trainer of claim 1, wherein the pressure sensor is arranged on an inner surface of at least one of the elongated members.

4. The tissue handling skills trainer of claim 1, wherein the pressure sensor is a piezoelectric sensor.

5. The tissue handling skills trainer of claim 1, wherein the trainer includes both an audio and visual indicator.

6. The tissue handling skills trainer of claim 1, further comprising a digital display operably connected to the microcontroller.

7. The tissue handling skills trainer of claim 1, further comprising a battery operably connected to the microcontroller.

8. A method for improving tissue handling skills of a user, comprising the step of using a tissue handling skills trainer to grasp a material, wherein the tissue handling skills trainer comprises
a forceps body having two elongated members hinged at one end,
at least one pressure sensor arranged on at least one of the elongated members,
a microcontroller electronically connected to the at least one pressure sensor, and
an audio and/or visual indicator electronically connected to the microcontroller, wherein the audio and/or visual indicator is triggered when the pressure sensor detects a pressure that exceeds a predetermined threshold.

9. The method of claim 8, wherein the material is a biological tissue.

10. The method of claim 9, wherein the tissue handling skills trainer is used while the user is performing a suturing procedure.
